(19) United States
(12) Patent Application Publication
(10) Pub. No.: US 2006/0270715 A1
(43) Pub. Date: Nov. 30, 2006

(54) Dosage forms of amlodipine and processes for their preparation

(76) Inventors: Romi Barat Singh, Uttar Pradesh (IN); Nidhi Singh, Delhi (IN); Vishnubhetla Nagaprasad, Andhra Pradesh (IN); Rajiv Malik, Wien (AT)

Correspondence Address: RANBAXY INC., 600 COLLEGE ROAD EAST, SUITE 2100, PRINCETON, NJ, 08540, US

(21) Appl. No.: 10/546,114

(22) PCT Filed: Feb. 27, 2004
(86) PCT No.: PCT/IB04/00512

§ 371 Date: Aug. 20, 2005

(30) Foreign Application Priority Data
Feb. 28, 2003 (IN) 201/DEL/2003

(51) Int. Cl. A61K 31/455; C07D 211/82

(52) U.S. Cl. 514/355; 546/315

(57) ABSTRACT

The technical field of the present invention relates to stable solid dosage forms of amlodipine base; and process of preparation thereof. In particular, it relates to solid dosage forms free of dicalcium phosphate. The stable amlodipine solid dosage form includes amlodipine base, microcrystalline cellulose, is substantially free of dicalcium phosphate, and has less than about 0.5% concentration (w/w) of Impurity D after three months at 40° C. and 75% relative humidity.

### TECHNICAL FIELD OF THE INVENTION

The technical field of the invention relates to stable solid dosage forms of amlodipine base and processes for their preparation. In particular, the solid dosage forms have reduced levels of {3-ethyl-5-methyl-2-[(2-aminoethoxy) methyl]-4-(2-chlorophenyl)-6-methylpyridine-3,5 dicarboxylate} (“impurity D”) and total impurities when free of dicalcium phosphate and include microcrystalline cellulose.

### BACKGROUND OF THE INVENTION

Amlodipine is a long acting calcium channel blocker marketed by Pfizer as amlodipine besylate under the trade name Norvasc®. It is available as oral tablets in strengths of 2.5 mg, 5 mg, and 10 mg, and is indicated for the treatment of hypertension, chronic stable angina and vasospastic angina. The inactive ingredients in the Norvasc® tablets include microcrystalline cellulose, dibasic calcium phosphate anhydrous, sodium starch glycolate, and magnesium stearate.

The preparation of amlodipine base is described in U.S. Pat. No. 4,572,909. Further, U.S. Pat. No. 4,879,303 discloses that free base compositions that include microcrystalline cellulose and dicalcium phosphate as diluents excessively stick to the tablet punches during processing and are not suitable in making solid dosage forms for peroral administration. The patent teaches that the amlodipine besylate-salt can be used to make solid dosage forms and those solid dosage forms can include microcrystalline cellulose and dibasic calcium phosphate. The microcrystalline cellulose is present at between about 62% (w/w) and about 76% (w/w) of the total dosage form composition. Subsequently, U.S. Patent Application No. 2003/0022922 discloses that to reduce the stickiness of the tablet, amlodipine free base should be incorporated into the tablet composition in the form of particulates having an average particle size of 150-350 μm; and a preferred excipient is a combination of calcium phosphate and microcrystalline cellulose. A capsule dosage form also is disclosed in this patent application as containing amlodipine base, microcrystalline cellulose, predried potato starch, and magnesium stearate. The microcrystalline cellulose makes up approximately 74% (w/w) of the capsule dosage form.

Amlodipine is highly hygroscopic and absorbs moisture, which leads to degradation. One of the major routes of degradation is via the catalytic oxidative process, which is pH dependent. The major related substances produced are {3-ethyl 5-methyl (4RS) 4-(2-chlorophenyl)-6-methyl-2-[[2-[[2-(methylcarbamoyl) benzoyl] amino] ethoxy] methyl]-1,4-dihydropyridine-3,5 dicarboxylate} (“Impurity B”); {3-ethyl-5-methyl-2-[(2-amninoethoxy) methyl]-4-(2-chlorophenyl)-6-methylpyridine-3,5 dicarboxylate} (“Impurity D”); and {3-ethyl 5-methyl (4RS) 4-(2-chlorophenyl)-2[[2-(1,3-dioxo-dihydro-2H-isoindol-2-yl) ethoxy]methyl]-6-methyl-1,4-dihydropyridine-3,5 dicarboxylate} (“Impurity A”), along with some unknown impurities. Being an unstable compound, amlodipine requires well-directed stability approaches to formulate pharmaceutical compositions with reasonable stability.

### SUMMARY OF THE INVENTION

In one general aspect there is provided a stable amlodipine solid dosage form that includes amnlodipine base, microcrystalline cellulose, is substantially free of dicalcium phosphate, and has less than about 0.5% concentration (w/w) of Impurity D after three months at 40° C. and 75% relative humidity.

Embodiments of the stable amlodipine solid dosage form may include one or more of the following features. For example, the stable solid dosage form may have less than about 2% concentration (w/w) of total impurities after three months at 40° C. and 75% relative humidity. The dosage form may have more than 80% (w/w) microcrystalline cellulose and may have more than 90% (w/w) microcrystalline cellulose.

The stable amnlodipine solid dosage form may further include one or more pharmaceutically inert excipients. The one or more pharmaceutically inert excipients may be selected from diluents, binders, desiccants, disintegrants, coloring agents, flavoring agents, stabilizers, surfactants, lubricants/glidants, plasticizers and preservatives. In particular, the desiccant may be colloidal silicon dioxide. The solid dosage form may be a tablet or a capsule.

In another general aspect there is provided a stable amlodipine solid dosage form that includes amlodipine base, microcrystalline cellulose, mannitol, is substantially free of dicalcium phosphate, and has less than about 0.75% concentration (w/w) of Impurity D after three months at 40° C. and 75% relative humidity.

Embodiments of the stable amlodipine solid dosage form may include one or more of the following features. For example, the stable solid dosage form may have less than about 2% concentration (w/w) of total impurities after three months at 40° C. and 75% relative humidity. The dosage form may have more than 60% (w/w) microcrystalline cellulose. The dosage form may have more than 20% (w/w) mannitol.

The dosage form may further include one or more pharmaceutically inert excipients. The one or more pharmaceutically inert excipients may be diluents, binders, desiccants, disintegrants, coloring agents, flavoring agents, stabilizers, surfactants, lubricants/glidants, plasticizers and preservatives. In particular, the desiccant may be colloidal silicon dioxide. The solid dosage form may be a tablet or a capsule.

In another general aspect there is provided a process for preparing a stable solid dosage form of amlodipine. The process includes blending an effective amount of amlodipine, microcrystalline cellulose and one or more pharmaceutically inert excipients; and processing into a solid dosage form. The dosage form is substantially free of dicalcium phosphate and has less than about 0.5% concentration (w/w) of Impurity D after three months at 40° C. and 75% relative humidity.

Embodiments of the process may include one or more of the following features. For example, the stable solid dosage form may have less than about 2% concentration (w/w) of total impurities after three months at 40° C. and 75% relative humidity. The dosage form may have more than 80% (w/w) microcrystalline cellulose. In particular, the dosage form may have more than 90% (w/w) microcrystalline cellulose.

The dosage form may further include one or more pharmaceutically inert excipients. The one or more pharmaceutically inert excipients may include diluents, binders, desiccants, disintegrants, coloring agents, flavoring agents, stabilizers, surfactants, lubricants/glidants, plasticizers and preservatives. In particular, the desiccant may be colloidal silicon dioxide. The solid dosage form prepared may be a tablet or a capsule. The process may further include granulating the blend.

In another general aspect there is provided a process for preparing a stable solid dosage form of amlodipine base. The process includes blending an effective amount of amlodipine base, microcrystalline cellulose, mannitol, and one or more pharmaceutically inert excipients; and processing into a solid dosage form. The dosage form is substantially free of dicalcium phosphate and has less than about 0.75% concentration (w/w) of Impurity D after three months at 40° C. and 75% relative humidity.

Embodiments of the process may include one or more of the following features. For example, the stable solid dosage form may have less than about 2% concentration (w/w) of total impurities after three months at 40° C. and 75% relative humidity. The dosage form may have more than 60% (w/w) microcrystalline cellulose. The dosage form may have more than 20% (w/w) mannitol.

The dosage form may include one or more pharmaceutically inert excipients. The one or more pharmaceutically inert excipients may be selected from diluents, binders, desiccants, disintegrants, coloring agents, flavoring agents, stabilizers, surfactants, lubricants/glidants, plasticizers and preservatives. In particular, the desiccant may be colloidal silicon dioxide. The solid dosage form thus prepared may a tablet or a capsule. The process may further include granulating the blend.

In another general aspect there is provided a method for the treatment of one or more symptoms selected from the group consisting of hypertension, chronic stable angina, and vasospastic angina in a mammal. The method includes administering to the mammal a stable amlodipine solid dosage form that includes amlodipine base, microcrystalline cellulose, is substantially free of dicalcium phosphate, and has less than about 0.5% concentration (w/w) of Impurity D after three months at 40° C. and 75% relative humidity.

Embodiments of the method of treatment may include any one or more of the features described above.

In another general aspect there is provided a stable amlodipine solid dosage form that includes amlodipine base, microcrystalline cellulose, is substantially free of dicalcium phosphate, and has a ratio of microcrystalline cellulose to amlodipine base of at least 24:1.

Embodiments of the dosage form may include any one or more of the features described above. For example, the dosage form may further include mannitol, sodium starch glycolate, colloidal silicon dioxide, and magnesium stearate.

The details of one or more embodiments of the invention are set forth in the description below. Other features and advantages of the invention will be apparent from the description and the claims.

### DETAILED DESCRIPTION OF THE INVENTION

The prior art literature discloses the use of dicalcium phosphate as one of the preferred excipients for amlodipine formulations. The inventors have discovered that the presence of dicalcium phosphate in the amlodipine formulation triggers the degradation of amlodipine, which is more pronounced at a pH below 6.0. Hence, removal of dicalcium phosphate from the composition provides more stable pharmaceutical compositions of amlodipine.

In our attempts to stabilize amlodipine in solid dosage forms we discovered that stability may be improved by replacing dicalcium phosphate with microcrystalline cellulose and mannitol. This is clearly evident from the stability data generated over a period of 3 months at 40° C. and 75% relative humidity on the basis of percentage concentrations of related substances. This stability data is provided below in Table 2, in terms of the % concentrations of related substances (w/w).

Hence, in one general aspect the inventors have developed a stable solid dosage form of amlodipine that includes an effective amount of amlodipine, an increased amount of microcrystalline cellulose relative to the prior art, is substantially free of dicalcium phosphate, and has less than about 0.5% concentration (w/w) of Impurity D after three months at 40° C. and 75% relative humidity. The dosage form also has less than about 0.2% concentration (w/w) of Impurity D after one month at 40° C. and 75% relative humidity. The stable solid dosage form has less than about 2% concentration (w/w) of total impurities after three months at 40° C. and 75% relative humidity. The dosage form also has less than about 0.6% concentration (w/w) of total impurities after one month at 40° C. and 75% relative humidity.

The inventors also have developed a stable solid dosage form that includes an effective amount of amnlodipine, microcrystalline cellulose and mannitol, but is substantially free of dicalcium phosphate, and has less than about 0.75% concentration (w/w) of Impurity D after three months at 40° C. and 75% relative humidity. The dosage form also has less than about 0.3% concentration (w/w) of Impurity D after one month at 40° C. and 75% relative humidity. The stable solid dosage form has less than about 2% concentration (w/w) of total impurities after three months at 40° C. and 75% relative humidity. The dosage form also has less than about 1% concentration (w/w) of total impurities after one month at 40° C. and 75% relative humidity.

In another general aspect, the inventors have developed a process for preparing stable solid dosage forms of amlodipine. The process includes (a) blending an effective amount of amlodipine, an increased amount of microcrystalline cellulose relative to the prior art, and pharmaceutically inert excipients; (b) optionally granulating the blend; and (c) processing the blend into a solid dosage form. The dosage forms thus prepared are substantially free of dicalcium phosphate. The process for preparing stable solid dosage forms of amlodipine instead may include: (a) blending an effective amount of amlodipine, microcrystalline cellulose, mannitol, and pharmaceutically inert excipients; (b) optionally granulating the blend; and (c) processing the blend into a solid dosage form. The dosage form is substantially free of dicalcium phosphate.

In yet another general aspect the inventors have developed a method for treating one or more symptoms selected from hypertension, chronic stable angina, and vasospastic angina in a mammal, by administering a stable solid dosage form of amlodipine that is substantially free of dicalcium phosphate.

The term “stable” as used herein refers to chemical stability of amlodipine in solid dosage forms and indicates presence of less than 2% w/w of related substances when stored at 40° C. and 75 percent relative humidity for 1 month. The stability is measured using HPLC to measure the presence of related substances.

Amlodipine as used herein is the free base and can be of any form including, for example, crystalline form I, crystalline form II, amorphous form, and mixtures thereof.

The term “dicalcium phosphate” as used herein includes anhydrous calcium phosphate, anhydrous dicalcium phosphate, dibasic calcium phosphate as well as hydrates and solvates thereof. Dicalcium phosphate is normally used as a diluent. The term “substantially free” as used herein refers to the use of dicalcium phosphate in a concentration less than that used as a diluent.

Microcrystalline cellulose is a white, odorless, tasteless, free flowing powder, and is widely accepted in the pharmaceutical industry as a universal diluent. It is purified; partially depolymerized alpha cellulose derived from purified specialty grades of wood pulp. There are various grades which differ in bulk density, particle size, and moisture content. Some of the commercially available grades of microcrystalline cellulose are Avicel®, Vivapur® and Tabulose®. When used without mannitol, the amount of microcrystalline cellulose is increased relative to the prior art, e.g., greater than 80% (w/w) and, more particularly, greater than 90% (w/w). When used with mannitol, the amount of microcrystalline cellulose is greater than about 60% (w/w).

Mannitol is a naturally occurring sugar alcohol having a cool taste and 50% sweetness compared to sucrose. It is non-hygroscopic, chemically inert and does not undergo the Maillard reaction, and therefore does not discolor in the presence of free amines. Mannitol is available as powder and free flowing granules, and is used widely in pharmaceutical preparations. The granular form is particularly useful in direct compression technique of preparing tablets. Some of the commercial grades are Mannogem®, Pearlitol® and Partech M®. The concentration of mannitol may vary from about 5% to about 80%, in particular it may vary from 20% to 60% by weight of the total uncoated tablet weight.

The term “solid dosage form” as used herein includes conventionally used dosage forms such as tablet, capsule and the like.

The term “pharmaceutically inert excipient” as used herein includes substances known in the art as diluents, binders, desiccants, disintegrants, coloring agents, flavoring agents, stabilizers, surfactants, lubricants/glidants, plasticizers and preservatives for pharmaceutical compositions. The excipients are selected based on the desired physical aspects of the final tablets; e.g., obtaining a tablet with desired hardness and friability, being rapidly dispersible and easily swallowed, etc. Further, the inert excipients may be so selected as to provide slow and/or controlled release of the amlodipine from the tablets.

Examples of disintegrants include sodium starch glycolate, croscarmellose sodium, crospovidone, low substituted hydroxypropyl cellulose, and the like.

Examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, and the like.

Examples of diluents include cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like.

Examples of lubricants and glidants include magnesium stearate, colloidal anhydrous silica, stearic acid, magnesium stearate, calcium stearate, talc, hydrogenated castor oil, sucrose esters of fatty acid, microcrystalline wax, yellow beeswax, white beeswax, and the like.

Examples of desiccants include colloidal silicon dioxide, silicon dioxide and the like.

Examples of surfactants include both non-ionic and ionic (Cationic, Anionic and Zwitterionic) surfactants suitable for use in pharmaceutical compositions. These include polyethoxylated fatty acids and its derivatives, for example polyethylene glycol 400 distearate, polyethylene glycol—20 dioleate, polyethylene glycol 4—150 mono dilaurate, polyethylene glycol—20 glyceryl stearate; alcohol—oil transesterification products, for example polyethylene glycol—6 corn oil; polyglycerized fatty acids, for example polyglyceryl—6 pentaoleate; propylene glycol fatty acid esters, for example propylene glycol monocaprylate; mono and diglycerides, for example glyceryl ricinoleate; sterol and sterol derivatives, for example sitosterol; sorbitan fatty acid esters and its derivatives, for example polyethylene glycol—20 sorbitan monooleate, sorbitan monolaurate; polyethylene glycol alkyl ether or phenols, for example polyethylene glycol—20 cetyl ether, polyethylene glycol—10—100 nonyl phenol; sugar esters, for example sucrose monopalmitate; polyoxyethylene—polyoxypropylene block copolymers known as “poloxamer”; ionic surfactants, for example sodium caproate, sodium glycocholate, soy lecithin, sodium stearyl filmarate, propylene glycol alginate, octyl sulfosuccinate disodium, palmitoyl carnitine; and the like.

Examples of plasticizers include polyethylene glycol, triethyl citrate, triacetin, diethyl phthalate, dibutyl sebacate and the like.

Examples of stabilizers include antioxidants, buffers, alkalizers, chelating agents and the like.

Examples of coloring agents include any FDA approved colors for oral use.

In one of the embodiments, stable amlodipine tablets are prepared by a process that includes the steps of: (a) blending an effective amount of amlodipine, microcrystalline cellulose, one or more disintegrants and, optionally, mannitol; (b) mixing the blend with one or more lubricants/glidants; (c) directly compressing the blend in a suitable tableting machine; and, optionally, (d) coating with one or more film forming polymers.

In another embodiment, stable amlodipine tablets are prepared by a process that includes the steps of: (a) blending an effective amount of amlodipine, microcrystalline cellulose, one or more disintegrants and, optionally, mannitol; (b) granulating the blend; (c) mixing the granules with lubricant/glidant; (d) compressing the blend in a suitable tabletting machine; and, optionally, (e) coating with one or more film forming polymers, if desired.

In yet another embodiment, stable amlodipine capsules are prepared by a process that includes the steps of: (a) blending an effective amount of amlodipine, microcrystalline cellulose, and, optionally, mannitol; (b) optionally granulating the blend; (c) mixing the granules/blend with one or more lubricants/glidants; and (d) filling the blend into suitable sized capsules.

Granulation may be carried out by wet granulation or dry granulation techniques. Coating may be performed by applying one or more film forming polymers, with or without other pharmaceutically inert excipients, as a solution/suspension using any conventional coating technique known in the art, such as spray coating in a conventional coating pan or fluidized bed processor; or dip coating.

The following examples illustrate the invention but should not be construed as limiting the scope of the invention.

### EXAMPLES

Amlodipine tablets according to the composition listed in Table 1, were prepared by using the following steps:
1. Appropriate amounts of amlodipine and all other ingredients were separately passed through suitable sized sieves.
2. Amlodipine, microcrystalline cellulose, sodium starch glycolate, colloidal silicon dioxide and dicalcium phosphate (only for Example 1) or mannitol (only for Example 3) were mixed together to form a uniform blend.
3. The blend of step 2 was lubricated by mixing with magnesium stearate.
4. The compression machine was adjusted to a fill weight of 400 mg.

5. The final lubricated blend of step 3 was directly compressed using suitable size punches to obtain compressed tablets.

TABLE 1
Composition of amlodipine tablets (Examples 1-3)

| | Weight (mg) | | |
|---|---|---|---|
| Ingredient | Example 1 | Example 2 | Example 3 |
| Amlodipine | 10 | 10 | 10 |
| Microcrystalline cellulose | 254 | 370 | 244 |
| Dicalcium phosphate | 116 | — | — |
| Mannitol | — | — | 126 |
| Sodium starch glycolate | 12 | 12 | 12 |
| Colloidal silicon dioxide | 4 | 4 | 4 |
| Magnesium stearate | 4 | 4 | 4 |

The tablets obtained above were subjected to stability evaluation over a period of 3 months at 40° C. and 75% relative humidity. Initially, after one month, and after the three months of aging, the tablets were evaluated for the presence of impurities using HPLC. The results of this measurement are listed as percentage (w/w) related substances in Table 2. These results indicate the clear role that dicalcium phosphate plays in the degradative reactions of amlodipine and the advantageous use of microcrystalline cellulose and mannitol in stabilization of amlodipine.

TABLE 2
Results of stability evaluation of amlodipine tablets (Examples
1-3) as percentage (w/w) related substances, over a period
of 3 months at 40° C. and 75% relative humidity.

| | Example 1 | | | Example 2 | | | Example 3 | | |
|---|---|---|---|---|---|---|---|---|---|
| Related | % Conc. (w/w) | | | % Conc. (w/w) | | | % Conc. (w/w) | | |
| Subs. (RS) | Initial | 1 M* | 3 M | Initial | 1 M | 3 M | Initial | 1 M | 3 M |
| Imp** D | 0.238 | 2.062 | 4.399 | 0.097 | 0.154 | 0.382 | 0.050 | 0.221 | 0.510 |
| Imp B | ND*** | ND | 0.020 | ND | ND | 0.025 | ND | ND | 0.012 |
| Imp A | ND | ND | ND | ND | ND | ND | ND | ND | ND |
| Others | 0.151 | 0.705 | 2.387 | 0.170 | 0.328 | 1.092 | 0.151 | 0.399 | 1.128 |
| Total | 0.389 | 2.767 | 6.806 | 0.267 | 0.482 | 1.499 | 0.201 | 0.620 | 1.650 |

*Month,
**Impurity,
***Not Detected

As can be seen from Table 2, Example 2 has less than about 0.5% concentration (w/w) of Impurity D after three months at 40° C. and 75% relative humidity and less than about 0.2% concentration (w/w) of Impurity D after one month at 40° C. and 75% relative humidity. Example 2 also has less than about 2% concentration (w/w) of total impurities after three 15 months at 40° C. and 75% relative humidity and less than about 0.6% concentration (w/w) of total impurities after one month at 40° C. and 75% relative humidity. Example 3 has less than about 0.75% concentration (w/w) of Impurity D after three months at 40° C. and 75% relative humidity and less than about 0.3% concentration (w/w) of Impurity D after one month at 40° C. and 75% relative humidity. Example 3 also has less than about 2% concentration (w/w) of total impurities after three months at 40° C. and 75% relative humidity and less than about 1% concentration (w/w) of total impurities after one month at 40° C. and 75% relative humidity.

Based on the results presented in Table 2, there appears to be a correlation between the formation of impurities and the amount of microcrystalline cellulose in the formulation, and the presence or absence of dicalcium phosphate in the formulation. The formulation of Example 1 had a ratio of microcrystalline cellulose to amlodipine of 25.4:1 with the presence of dicalcium phosphate in the tablet. This formulation, however, had significantly higher levels of impurity D and total impurities initially, after one month, and after three months. In contrast, the formulation of Example 2 had a ratio of microcrystalline cellulose to amlodipine of 37:1 without the presence of dicalcium phosphate in the tablet. Relative to the formulation of Example 1, this formulation had significantly lower levels of impurity D and total impurities initially, after one month, and after three months. Similarly, the formulation of Example 3 had a ratio of microcrystalline cellulose to amlodipine of 24.4:1 without the presence of dicalcium phosphate in the tablet. Relative to the formulation of Example 1, this formulation had significantly lower levels of impurity D and total impurities initially, after one month, and after three months. Thus, the data in Table 2 indicates that the stability of dosage forms of amlodipine base can be improved based on the amount and ratio of microcrystalline cellulose to amlodipine base in the formulation and the presence or absence of dicalcium phosphate in the formulation.

While several particular formulations have been described above, it will be apparent that various modifications and combinations of the formulations detailed in the text can be made without departing from the spirit and scope of the invention. For example although the tablet dosage form has been prepared, other conventional solid dosage forms, such as capsules, can also be prepared using compositions similar to those disclosed herein.

Similarly, using the disclosure contained herein, dosage forms of amlodipine can be made that are one or more of bioequivalent to the reference listed drug product or have suitable in vitro dissolution profiles. For example, the following dosage forms have been made and tested:

TABLE 3
Compositions of amlodipine tablets (Examples 4-6)

| | Weight (mg) per Tablet | | |
|---|---|---|---|
| Ingredient | Example 4 | Example 5 | Example 6 |
| Amlodipine* | 2.50 | 5.00 | 10.00 |
| Microcrystalline cellulose NF | 60.50 | 121.0 | 242.0 |
| Mannitol USP | 31.50 | 63.00 | 126.0 |
| Sodium starch glycolate NF | 3.00 | 6.00 | 12.0 |
| Colloidal silicon dioxide NF | 1.00 | 2.00 | 4.00 |
| Magnesium stearate NF | 1.50 | 3.00 | 6.00 |
| Tablet Weight | 100.00 | 200.00 | 400.00 |

*This quantity is calculated based on 100% w/w assay on anhydrous base and no water content.

Similarly, although a direct compression method has been used in preparing tablets of Examples 1 through 3, other conventional methods can also be used. For example, appropriately sieved amlodipine may be blended with other ingredients and a part of magnesium stearate, and compacted in a roller compacter to produce granules. The granules may then be sieved and lubricated with the remaining amount of magnesium stearate and compressed into suitable sized tablets. Alternatively, appropriately sieved amlodipine and other ingredients (except magnesium stearate) may be blended into a wet mass using a granulating fluid. Suitable sized granules can then be prepared in granulator. The granules may be dried, lubricated by mixing with magnesium stearate, and compressed into suitable sized tablets.

Similarly, although amlodipine base has been used in preparing the tablets of Examples 1 through 3, tablets may be prepared using compositions similar to those described for amlodipine base for pharmaceutically acceptable salts of amlodipine, such as amlodipine besylate and amlodipine mesylate, as well as any and all pharmaceutically acceptable salts of amlodipine that are incompatible with dicalcium phosphate.

We claim:

1. A stable amlodipine solid dosage form, the dosage form comprising amlodipine base, microcrystalline cellulose, being substantially free of dicalcium phosphate, and having less than about 0.5% concentration (w/w) of Impurity D after three months at 40° C. and 75% relative humidity.

2. The stable amlodipine solid dosage form according to claim 1, wherein the stable solid dosage form has less than about 2% concentration (w/w) of total impurities after three months at 40° C. and 75% relative humidity.

3. The stable amlodipine solid dosage form according to claim 1, wherein the dosage form comprises more than 80% (w/w) microcrystalline cellulose.

4. The stable amlodipine solid dosage form according to claim 1, wherein the dosage form comprises more than 90% (w/w) microcrystalline cellulose.

5. The stable amlodipine solid dosage form according to claim 1, further comprising one or more pharmaceutically inert excipients.

6. The stable amlodipine solid dosage form according to claim 5, wherein the one or more pharmaceutically inert excipients are selected from diluents, binders, desiccants, disintegrants, coloring agents, flavoring agents, stabilizers, surfactants, lubricants/glidants, plasticizers and preservatives.

7. The stable amlodipine solid dosage form according to claim 6, wherein the desiccant comprises colloidal silicon dioxide.

8. The stable amlodipine solid dosage form according to claim 1, wherein the solid dosage form comprises a tablet or a capsule.

9. A stable amlodipine solid dosage form, the dosage form comprising amlodipine base, microcrystalline cellulose, mannitol, being substantially free of dicalcium phosphate, and having less than about 0.75% concentration (w/w) of Impurity D after three months at 40° C. and 75% relative humidity.

10. The stable amlodipine solid dosage form according to claim 9, wherein the stable solid dosage form has less than about 2% concentration (w/w) of total impurities after three months at 40° C. and 75% relative humidity.

11. The stable amlodipine solid dosage form according to claim 9, wherein the dosage form comprises more than 60% (w/w) microcrystalline cellulose.

12. The stable amlodipine solid dosage form according to claim 9, wherein the dosage form comprises more than 20% (w/w) mannitol.

13. The stable amlodipine solid dosage form according to claim 9, further comprising one or more pharmaceutically inert excipients.

14. The stable amlodipine solid dosage form according to claim 9, wherein the one or more pharmaceutically inert excipients are selected from diluents, binders, desiccants, disintegrants, coloring agents, flavoring agents, stabilizers, surfactants, lubricants/glidants, plasticizers and preservatives.

15. The stable amlodipine solid dosage form according to claim 14, wherein the desiccant comprises colloidal silicon dioxide.

16. The stable amlodipine solid dosage form according to claim 9, wherein the solid dosage form comprises a tablet or a capsule.

17. A process for the preparation of a stable solid dosage form of amlodipine base comprising the steps of:
blending an effective amount of amlodipine base, microcrystalline cellulose and one or more pharmaceutically inert excipients; and
processing into a solid dosage form;
the dosage form being substantially free of dicalcium phosphate, and having less than about 0.5% concentration (w/w) of Impurity D after three months at 40° C. and 75% relative humidity.

18. The process according to claim 17, wherein the stable solid dosage form has less than about 2% concentration (w/w) of total impurities after three months at 40° C. and 75% relative humidity.

19. The process according to claim 17, wherein the dosage form comprises more than 80% (w/w) microcrystalline cellulose.

20. The process according to claim 17, wherein the dosage form comprises more than 90% (w/w) microcrystalline cellulose.

21. The process according to claim 17, further comprising one or more pharmaceutically inert excipients.

22. The process according to claim 21, wherein the one or more pharmaceutically inert excipients are selected from diluents, binders, desiccants, disintegrants, coloring agents, flavoring agents, stabilizers, surfactants, lubricants/glidants, plasticizers and preservatives.

23. The process according to claim 22, wherein the desiccant comprises colloidal silicon dioxide.

24. The process according to claim 23, wherein the solid dosage form comprises a tablet or a capsule.

25. The process according to claim 23, further comprising granulating the blend.

26. A process for the preparation of a stable solid dosage form of amlodipine base comprising the steps of:
blending an effective amount of amlodipine base, microcrystalline cellulose, mannitol, and one or more pharmaceutically inert excipients; and
processing into a solid dosage form;
the dosage form being substantially free of dicalcium phosphate, and having less than about 0.75% concentration (w/w) of Impurity D after three months at 40° C. and 75% relative humidity.

27. The process according to claim 26, wherein the stable solid dosage form has less than about 2% concentration (w/w) of total impurities after three months at 40° C. and 75% relative humidity.

28. The process according to claim 26, wherein the dosage form comprises more than 60% (w/w) microcrystalline cellulose.

29. The process according to claim 26, wherein the dosage form comprises more than 20% (w/w) mannitol.

30. The process according to claim 26, further comprising one or more pharmaceutically inert excipients.

31. The process according to claim 30, wherein the one or more pharmaceutically inert excipients are selected from diluents, binders, desiccants, disintegrants, coloring agents, flavoring agents, stabilizers, surfactants, lubricants/glidants, plasticizers and preservatives.

32. The process according to claim 31, wherein the desiccant comprises colloidal silicon dioxide.

33. The process according to claim 26, wherein the solid dosage form comprises a tablet or a capsule.

34. The process according to claim 26, further comprising granulating the blend.

35. A method for the treatment of one or more symptoms selected from the group consisting of hypertension, chronic stable angina, and vasospastic angina in a mammal, by administering to the said mammal a stable amlodipine solid dosage form comprising amlodipine base, microcrystalline cellulose, being substantially free of dicalcium phosphate, and having less than about 0.5% concentration (w/w) of Impurity D after three months at 40° C. and 75% relative humidity.

36. A stable amlodipine solid dosage form, the dosage form comprising amlodipine base, microcrystalline cellulose, being substantially free of dicalcium phosphate, and having a ratio of microcrystalline cellulose to amlodipine base of at least 24:1.

37. The stable amlodipine solid dosage form of claim 36, the dosage form further comprising mannitol, sodium starch glycolate, colloidal silicon dioxide, and magnesium stearate.
